(19) United States
(12) Patent Application Publication
(10) Pub. No.: US 2025/0331765 A1
(43) Pub. Date: Oct. 30, 2025

(54) METHODS AND SYSTEMS FOR NON-INVASIVE CHARACTERIZATION OF MECHANORECEPTORS

(71) Applicant: University of Rochester, Rochester, NY (US)

(72) Inventors: Peter D. Creigh, Rochester, NY (US); Steffen Behrens-Spraggins, Rochester, NY (US); David N. Herrmann, Rochester, NY (US); Jiebo Luo, Rochester, NY (US); Ashwin Ramesh, Rochester, NY (US); Yuan Yao, Rochester, NY (US)

(73) Assignee: University of Rochester, Rochester, NY (US)

(21) Appl. No.: 19/191,509

(22) Filed: Apr. 28, 2025

Related U.S. Application Data
(60) Provisional application No. 63/640,035, filed on Apr. 29, 2024.

(51) Int. Cl. A61B 5/00; G06T 7/00; G06V 10/25; G06V 20/69; G16H 30/40

(52) U.S. Cl. CPC A61B 5/4005; A61B 5/0082; A61B 5/441; A61B 5/7485; G06T 7/0012; G06V 10/25; G06V 20/69; G16H 30/40; A61B 2576/02; G06T 2207/10016; G06T 2207/10056; G06T 2207/30088; G06V 2201/03

(57) ABSTRACT

This disclosure describes novel methods and systems enabled by machine learning models for automated, non-invasive, and in vivo quantification of mechanoreceptors. The disclosed methods and systems can be used for determining or monitoring a condition associated with peripheral nervous system disorders, such as sensory neuropathies, sensory neuronopathies, sensorimotor neuropathies, and small fiber neuropathies.

### CROSS-REFERENCE TO RELATED APPLICATIONS

This application is entitled to priority pursuant to 35 U.S.C. § 119(e) to U.S. Provisional Application No. 63/640,035, filed on Apr. 29, 2024. The content of the application is incorporated herein by reference in its entirety.

### FIELD OF THE INVENTION

This disclosure generally relates to methods and systems for non-invasive characterization of mechanoreceptors.

### BACKGROUND OF THE INVENTION

Peripheral neuropathy is a common neurologic disorder involving injury to multiple peripheral nerves. There are numerous etiologies of peripheral neuropathy, including acquired and genetic processes. Validated objective markers that can detect and track peripheral nerve damage in these diseases are essential for clinical care and experimental therapeutic development. However, most existing validated markers of peripheral neuropathy are invasive or painful, limiting their utility.

Skin biopsy studies have identified Meissner's corpuscles (MCs) density as a sensitive marker of sensory nerve involvement in peripheral neuropathies. MCs are the main touch-pressure sensory mechanoreceptors in glabrous skin (hands and feet), and MC densities are reduced in peripheral neuropathy. However, MC density has not been widely used as a neuropathy marker, as glabrous skin biopsies are invasive and unsuitable for serial monitoring.

Therefore, there is a critical need to devise improved methods and systems for non-invasive characterization of mechanoreceptors.

### SUMMARY OF THE INVENTION

This disclosure addresses the need mentioned above in a number of aspects. In one aspect, this disclosure provides a method of determining one or more characteristics of mechanoreceptors in a region of interest on skin of a patient. In some embodiments, the method comprises: (a) inputting image data comprising an image stack obtained from the region of interest on the skin of the patient; (b) separating the stack image into a sequence of images; (c) detecting mechanoreceptors on each of the sequence of images and outlining the detected mechanoreceptors on each of the sequence of images using a trained annotator; (d) associating each of the outlined mechanoreceptors on an image of the sequence of images with another outlined mechanoreceptor on a neighboring image of the sequence of images to reconstitute three-dimensional shapes of the outlined mechanoreceptors across a subset of the images; and (e) determining, based on the three-dimensional shapes of the outlined mechanoreceptors, one or more characteristics of the mechanoreceptors in the region of interest of the skin of the patient, wherein the one or more characteristics comprise count, density, size, morphology, or a combination thereof.

In some embodiments, the step of detecting mechanoreceptors is performed by a YOLOv4 object detection model. In some embodiments, the step of outlining comprises placing bounding boxes around the outlined mechanoreceptors.

In some embodiments, the step of associating is performed based on intersection over union across the subset of the images.

In some embodiments, the step of associating comprises filtering out the associated outlined mechanoreceptors that are shorter than a threshold length.

In some embodiments, the step of associating comprises associating the outlined mechanoreceptors across the subset of images only if the outlined mechanoreceptors attributable to the same mechanoreceptor are present on at least a threshold number of consecutive images of the sequence of images. In some embodiments, the threshold number of the consecutive images is 2 to 20. In some embodiments, the threshold number of the consecutive images is 3.

In some embodiments, the step of associating comprises interpolating intermediate missing outlined mechanoreceptors in both size and location along an otherwise contiguous three-dimensional shape of the outlined mechanoreceptors.

In some embodiments, the sequence of images comprise 10 to 50 images (e.g., 35 images). In some embodiments, two neighboring images of the sequence of images are about 2 μm to 20 μm (e.g., 7 μm) apart.

In some embodiments, the mechanoreceptors comprise Meissner's corpuscles. In some embodiments, the step of determining the one or more characteristics of the mechanoreceptors comprises quantifying a density of the Meissner's corpuscles. In some embodiments, the step of determining the one or more characteristics of the mechanoreceptors comprises quantifying a size of the Meissner's corpuscles.

In some embodiments, the method further comprises determining the one or more characteristics of the mechanoreceptors over time to monitor a change in the one or more characteristics of the mechanoreceptors.

In some embodiments, the image data is obtained by confocal microscopy. In some embodiments, the confocal microscopy comprises in vivo reflectance confocal microscopy (RCM).

In another aspect, this disclosure also provides a method of determining or monitoring a condition in the patient based on one or more characteristics of mechanoreceptors in a region of interest on skin of a patient. In some embodiments, the method comprises determining one or more characteristics of mechanoreceptors in the region of interest on the skin of the patient according to the method described herein; and determining a condition in the patient based on the determined one or more characteristics of the mechanoreceptors.

In another aspect, this disclosure also provides a system for determining one or more characteristics of mechanoreceptors in a region of interest on skin of a patient. In some embodiments, the system comprises one or more processors configured to: (i) input image data comprising an image stack obtained from the region of interest on the skin of the patient; (ii) separate the stack image into a sequence of images; (iii) detect mechanoreceptors on each of the sequence of images and outline the detected mechanoreceptors on each of the sequence of images using a trained annotator; (iv) associate each of the outlined mechanoreceptors on an image of the sequence of images with another outlined mechanoreceptor on a neighboring image of the sequence of images to reconstitute three-dimensional shapes of the outlined mechanoreceptors across a subset of the images; and (v) determine, based on the three-dimensional shapes of the outlined mechanoreceptors, one or more characteristics of the mechanoreceptors in the region of interest of the skin of the patient, wherein the one or more characteristics comprise count, density, size, morphology, or a combination thereof.

In some embodiments, the step of detecting mechanoreceptors is performed by a YOLOv4 object detection model. In some embodiments, the step of outlining comprises placing bounding boxes around the outlined mechanoreceptors.

In some embodiments, the step of associating is performed based on intersection over union across the subset of the images.

In some embodiments, the step of associating comprises filtering out the associated outlined mechanoreceptors that are shorter than a threshold length.

In some embodiments, the step of associating comprises associating the outlined mechanoreceptors across the subset of images only if the outlined mechanoreceptors attributable to the same mechanoreceptor are present on at least a threshold number of consecutive images of the sequence of images. In some embodiments, the threshold number of the consecutive images is 2 to 20. In some embodiments, the threshold number of the consecutive images is 3.

In some embodiments, the step of associating comprises interpolating intermediate missing outlined mechanoreceptors in both size and location along an otherwise contiguous three-dimensional shape of the outlined mechanoreceptors.

In some embodiments, the sequence of images comprise 10 to 50 images (e.g., 35 images). In some embodiments, two neighboring images of the sequence of images are about 2 μm to 20 μm (e.g., 7 μm) apart.

In some embodiments, the mechanoreceptors comprise Meissner's corpuscles. In some embodiments, the step of determining the one or more characteristics of the mechanoreceptors comprises quantifying a density of the Meissner's corpuscles. In some embodiments, the step of determining the one or more characteristics of the mechanoreceptors comprises quantifying a size of the Meissner's corpuscles.

In some embodiments, the one or more processors are further configured to determine the one or more characteristics of the mechanoreceptors over time to monitor a change in the one or more characteristics of the mechanoreceptors.

In some embodiments, the image data is obtained by confocal microscopy. In some embodiments, the confocal microscopy comprises in vivo reflectance confocal microscopy (RCM).

In yet another aspect, this disclosure provides a system of determining or monitoring a condition in the patient based on one or more characteristics of mechanoreceptors in a region of interest on skin of a patient. In some embodiments, the system comprises one or more processors configured to: determine one or more characteristics of mechanoreceptors in the region of interest on the skin of the patient according to the system described herein; and determine a condition in the patient based on the determined one or more characteristics of the mechanoreceptors.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combinations of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIG. 1 shows sampling of reflectance confocal microscopy (RCM) on the skin of a subject.

FIG. 2 shows an example process of data annotation and model training for detecting and annotating Meissner's corpuscles (MCs).

FIG. 3 shows an example process of prediction and postprocessing of MCs.

FIG. 4 shows an example computing architecture for implementing the disclosed methods.

### DETAILED DESCRIPTION OF THE INVENTION

This disclosure describes novel methods and systems enabled by machine learning models for automated, non-invasive, and in vivo quantification of mechanoreceptors. The disclosed methods and systems can be used for determining or monitoring a condition associated with peripheral nervous system disorders, such as sensory neuropathies, sensory neuronopathies, sensorimotor neuropathies, and small fiber neuropathies.

### Methods for Non-Invasive Characterization of Mechanoreceptors

Accordingly, in one aspect, this disclosure provides a method of determining one or more characteristics of mechanoreceptors in a region of interest on skin of a patient. In some embodiments, the method may include: (a) inputting image data comprising an image stack obtained from the region of interest on the skin of the patient; (b) separating the stack image into a sequence of images; (c) detecting mechanoreceptors on each of the sequence of images and outlining the detected mechanoreceptors (for example, with boxes) on each of the sequence of images using a trained annotator; (d) associating each of the outlined mechanoreceptors (for example, associating the boxes) on an image of the sequence of images with another outlined mechanoreceptor on a neighboring image of the sequence of images to reconstitute three-dimensional shapes of the outlined mechanoreceptors across a subset of the images; and (e) determining, based on the three-dimensional shapes of the outlined mechanoreceptors, one or more characteristics of the mechanoreceptors in the region of interest of the skin of the patient, wherein the one or more characteristics may include count, density, size, morphology, or a combination thereof.

In some embodiments, the mechanoreceptors may include Meissner's corpuscles (MCs).

In some embodiments, the image data may include two-dimensional or three-dimensional imaging data. In some embodiments, the image data may include time-lapse imaging data, a video, or live video streaming data. As used herein, the term “image” or “images” refers to single or multiple frames of still or animated images, video clips, video streams, etc. Preprocessing may include detecting a image in the image of the subject by the user device. Preprocessing may also include cropping, resizing, gradation conversion, median filtering, histogram equalization, or size-normalized image processing. In some embodiments, the method may include resizing the photo or the videos according to a threshold value (e.g., maximum size in kilobytes, megabytes or gigabytes, maximum or minimum resolution in dots per inch (DPI) or pixels per inch (PPI)).

In some embodiments, the sequence of images may include 10 to 50 images (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 images). In some embodiments, the sequence of images may include 35 images.

In some embodiments, two neighboring images of the sequence of images are about 2 μm to 20 μm (e.g., 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, or 20 μm) apart. In some embodiments, two neighboring images of the sequence of images are about 7 μm apart.

In some embodiments, MCs have one or more features, such as location within tips of dermal papillae of glabrous skin, absence of similar profiles in hairy skin, dimensions in a range of reference dimensions of MCs in skin biopsy specimens, orientation with a long axis perpendicular to a dermal-epidermal junction, presence of one to two MCs per-dermal papilla, presence of an encapsulated structure with an internal lobulated axonal architecture, or a combination thereof.

In some embodiments, the step of determining one or more characteristics of the mechanoreceptors may include quantifying the density of the MCs. In some embodiments, the step of determining one or more characteristics of the mechanoreceptors may include quantifying the size of the MCs. In some embodiments, the step of identifying the mechanoreceptors and the step of determining one or more characteristics of the mechanoreceptors are performed over time to monitor a change of one or more characteristics of the mechanoreceptors.

In one example, to identify MCs, dermal papillae can be examined for profiles with morphologic characteristics of MCs, as defined by histological and immunohistochemical studies. Criteria for identifying a profile as an MC on in vivo reflectance confocal microscopy (RCM) may include: location within the tips of dermal papillae of glabrous skin, and the absence of similar profiles in hairy skin; dimensions in the range of those reported for MCs in skin biopsy specimens (mean 80×30 μm); orientation with the long axis approximately perpendicular to the dermal-epidermal junction; the presence of 0, 1, or occasionally 2 MCs per-dermal papilla; and/or the presence of an encapsulated structure, with an internal lobulated axonal architecture.

MCs can appear as heterogeneous bright structures within dermal papillae which appears as dark “pits”. Mean MC density in controls may be about 12±5.3/mm² (digit V) and 5.1±2.2/mm² at the thenar eminence. MC density in sensory neuropathies (SN) is lower than controls at digit V (about 2.8±5.7/mm², p=0.01), and the thenar eminence (about 1.4±1.1/mm², p=0.004). In some embodiments, MCs are absent in a sensory neuronopathy, and milder reductions in MC density can be seen among diabetic and HIV subjects.

In some embodiments, the image data is obtained by confocal microscopy. In some embodiments, the confocal microscopy may include in vivo reflectance confocal microscopy (RCM).

In some embodiments, MCs can be visualized and quantitated in controls and sensory neuropathies using in vivo RCM. In vivo RCM of MCs has a potential for non-invasive detection and monitoring of sensory neuropathies. For sensory neuropathies (SN), RCM imaging can be used as a tool to evaluate MCs which subserve sensory function (not motor functions). Many peripheral neuropathies are mixed and have sensory and motor components, i.e., sensorimotor neuropathies.

The terms “patient,” “subject,” “host,” and “individual” are used interchangeably herein and refer to any subject, particularly a vertebrate subject, and even more particularly a mammalian subject, for whom therapy or prophylaxis is desired. Suitable vertebrate animals that fall within the scope of the invention include, but are not restricted to, any member of the subphylum Chordata including primates (e.g., humans, monkeys and apes, and includes species of monkeys such from the genus Macaca (e.g., cynomolgus monkeys such as Macaca fascicularis, and/or rhesus monkeys (Macaca mulatta)) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes)), rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards etc.), and fish. In some embodiments, a subject is a human with a peripheral nervous system disorder.

In some embodiments, the object detection model or the annotator may include a machine learning model. In some embodiments, the trained annotator comprises a trained machine learning model.

As used herein, a “machine learning model,” a “model,” or a “classifier” refers to a set of algorithmic routines and parameters that can predict an output(s) for a process input based on a set of input features, with or without being explicitly programmed. A structure of the software routines (e.g., number of subroutines and relation between them) and/or the values of the parameters can be determined in a training process, which can use actual results of the process that is being modeled. Such systems or models are understood to be necessarily rooted in computer technology, and in fact, cannot be implemented or even exist in the absence of computing technology. While machine learning systems utilize various types of statistical analyses, machine learning systems are distinguished from statistical analyses by virtue of the ability to learn without explicit programming and being rooted in computer technology. A neural network or an artificial neural network is a set of algorithms used in machine learning for modeling the data using graphs of neurons. Any network structure may be used. Any number of layers, nodes within layers, types of nodes (activations), types of layers, interconnections, learnable parameters, and/or other network architectures may be used. Machine training uses the defined architecture, training data, and optimization to learn values of the learnable parameters of the architecture based on the samples and ground truth of training data.

A typical machine learning pipeline may include building a machine learning model from a sample dataset (referred to as a “training set”), evaluating the model against one or more additional sample datasets (referred to as a “validation set” and/or a “test set”) to decide whether to keep the model and to benchmark how good the model is, and using the model in “production” to make predictions or decisions against live input data captured by an application service. For training the model to be applied as a machine-learned model, training data is acquired and stored in a database or memory. The training data is acquired by aggregation, mining, loading from a publicly or privately formed collection, transfer, and/or access. Ten, hundreds, or thousands of samples of training data are acquired. The samples are from scans of different patients and/or phantoms. Simulation may be used to form the training data. The training data includes the desired output (ground truth), such as segmentation, and the input, such as protocol data and imaging data.

In some embodiments, the training set will be used to create a single classifier using any now or hereafter known methods. In other embodiments, a plurality of training sets will be created to generate a plurality of corresponding classifiers. Each of the plurality of classifiers can be generated based on the same or different learning algorithm that utilizes the same or different features in the corresponding one of the pluralities of training sets. For example, each of the plurality of neural network models can be trained on a training set classified on sequence type, view type, anatomy type and/or other image classifying data as discussed in conjunction with the disclosure.

Once trained, the machine-learned or trained classifier is stored for later application. The training determines the values of the learnable parameters of the network. The network architecture, values of non-learnable parameters, and values of the learnable parameters are stored as the machine-learned network. Once stored, the machine-learned network may be fixed. The same machine-learned network may be applied to different patients, different scanners, and/or with different imaging protocols for the scanning. The machine-learned network may be updated. As additional training data is acquired, such as through application of the network for patients and corrections by experts to that output, the additional training data may be used to re-train or update the training.

For the machine learning model, input data structures of subreads can be used for the training. The training is performed by optimizing parameters of the model based on outputs of the model matching or not matching corresponding labels of the first labels and optionally the second labels when the first plurality of first data structures and optionally the second plurality of second data structures are input to the model. In some embodiments, the output of the model may include a probability of being in each of a plurality of states. The state with the highest probability can be taken as the state.

In some embodiments, the machine learning model may further include a supervised learning model. Supervised learning models may include different approaches and algorithms including analytical learning, artificial neural network, backpropagation, boosting (meta-algorithm), Bayesian statistics, case-based reasoning, decision tree learning, inductive logic programming, Gaussian process regression, genetic programming, group method of data handling, kernel estimators, learning automata, learning classifier systems, minimum message length (decision trees, decision graphs, etc.), multilinear subspace learning, naive Bayes classifier, maximum entropy classifier, conditional random field, Nearest Neighbor Algorithm, probably approximately correct learning (PAC) learning, ripple down rules, a knowledge acquisition methodology, symbolic machine learning algorithms, subsymbolic machine learning algorithms, support vector machines, Minimum Complexity Machines (MCM), random forests, ensembles of classifiers, ordinal classification, data pre-processing, handling imbalanced datasets, statistical relational learning, or Proaftn, a multicriteria classification algorithm, linear regression, logistic regression, deep recurrent neural network (e.g., long short term memory, LSTM), Bayes classifier, hidden Markov model (HMM), linear discriminant analysis (LDA), k-means clustering, density-based spatial clustering of applications with noise (DBSCAN), random forest algorithm, support vector machine (SVM), or any model described herein.

In some embodiments, the classifier may include a supervised or unsupervised Machine Learning or Deep Learning algorithm, Logistic Regression, Naive Bayes, Support Vector Machine, Decision Tree, Random Forest, Gradient Boosting, Regularizing Gradient Boosting, K-Nearest Neighbors, a continuous regression approach, Ridge Regression, Kernel Ridge Regression, Support Vector Regression, deep learning approach, Neural Networks, Convolutional Neural Network (CNNs), Recurrent Neural Networks (RNNs), Gated Recurrent Units (GRUs), Long Short Term Memory Networks (LSTMs), Generative Models, Generative Adversarial Networks (GANs), Deep Belief Networks (DBNs), Feedforward Neural Networks, Autoencoders, Variational Autoencoders, Normalizing Flow Models, Deniosing Diffusion Probabilistic Models (DDPMs), Score Based Generative Models (SGMs), Radial Basis Function Networks (RBFNs), Multilayer Perceptrons (MLPs), Stochastic Neural Networks, or any combination thereof.

In some embodiments, the model may include a convolutional neural network (CNN). The CNN may include a set of convolutional filters configured to filter the first plurality of data structures and, optionally, the second plurality of data structures. The filter may be any filter described herein. The number of filters for each layer may be from 10 to 20, 20 to 30, 30 to 40, 40 to 50, 50 to 60, 60 to 70, 70 to 80, 80 to 90, 90 to 100, 100 to 150, 150 to 200, or more. The kernel size for the filters can be 2, 3, 4, 5, 6, 7, 8, 9, 10, 11, 12, 13, 14, 15, from 15 to 20, from 20 to 30, from 30 to 40, or more. CNN may include an input layer configured to receive the filtered first plurality of data structures and, optionally, the filtered second plurality of data structures. CNN may also include a plurality of hidden layers, including a plurality of nodes. The first layer of the plurality of hidden layers is coupled to the input layer. CNN may further include an output layer coupled to a last layer of the plurality of hidden layers and configured to output an output data structure. The output data structure may include the properties.

In some embodiments, the method may include identifying mechanoreceptors (e.g., MCs) using a classifier.

As used herein, the term “classifiers” refers generally to various types of classifier frameworks, such as neural network classifiers, hierarchical classifiers, ensemble classifiers, etc. In addition, a classifier design can include a multiplicity of classifiers that attempt to partition data into two groups, either organized hierarchically or run in parallel, and then combined to find the best classification. Further, a classifier can include ensemble classifiers wherein a large number of classifiers all attempting to perform the same classification task are learned, but trained with different data/variables/parameters, and then combined to produce a final classification label. The classification methods implemented may be “black boxes” that are unable to explain their prediction to a user (which is the case if classifiers are built using neural networks, for example). The classification methods may be “white boxes” that are in a human-readable form (which is the case if classifiers are built using decision trees, for example). In other embodiments, the classification models may be “gray boxes” that can partially explain how solutions are derived (e.g., a combination of “white box” and “black box” type classifiers).

As used herein, the term “classification” refers to any number or other characters that are associated with a particular property of a sample. The classification can be binary (e.g., positive or negative) or have more levels of classification (e.g., a scale from 1 to 10 or 0 to 1). The term “cutoff” or “threshold” refers to a predetermined number used in an operation. For example, a cutoff value can refer to a classification score as used above. A threshold value may be a value above or below which a particular classification applies. Either of these terms can be used in either of these contexts.

In some embodiments, the step of detecting mechanoreceptors is performed by an object detection model. In some embodiments, the object detection model is a YOLOv4 object detection model. YOLOv4, or You Only Look Once version 4, is a real-time object detection model. It is a one-stage network made up of three parts: neck, backbone, and head. YOLOv4 is an advanced deep neural network that can detect objects from digital images (M. Ning, et al., 2021 IEEE 45^{th} Annual Computers, Software, and Applications Conference (COMPSAC), Madrid, Spain, 2021, pp. 31-36). It is one of the fastest and most accurate methods available. YOLOv4 is designed to address the limitations of earlier YOLO versions, such as YOLOv3. YOLOv4 uses new features such as: WRC, CSP, CmBN, SAT, Mish activation, Mosaic data augmentation, CmBN, DropBlock regularization, CIoU loss. OLOv4 can be trained using a conventional GPU. The backbone can be a pretrained convolutional neural network, such as CSPDarkNet53 or VGG16, trained on COCO or ImageNet data sets.

In some embodiments, the step of outlining may include placing bounding boxes around the outlined mechanoreceptors. In some embodiments, boxes may have any shape, such as a circle, a polygon, a ring shape, a square, a rectangle, an oval shape, an ellipse shape, a regular polygon or irregular polygon, or a combination thereof. The regular or irregular polygons may include a triangle, square, pentagon, hexagon, septagon, octagon, nonagon, decagon, or any combination thereof.

In some embodiments, the step of associating is performed based on intersection over union across the subset of the images. Intersection over Union (IoU) is a metric used to measure the accuracy of object detection algorithms. It calculates the amount of overlap between a predicted bounding box and a ground truth bounding box. IoU is a popular metric for computing localization errors and measuring localization accuracy in object detection models. It's also used to evaluate the accuracy of annotation and segmentation algorithms. To calculate the union area of two boxes, add the area of both boxes and subtract the area of their intersection. IoU is a value between 0 and 1. The IoU threshold acts as a gatekeeper, classifying predicted bounding boxes as true positives if they pass the threshold and false positives if they fall below it.

In some embodiments, the step of associating comprises filtering out the associated outlined mechanoreceptors that are shorter than a threshold length. In some embodiments, the threshold length is about 20 μm to about 1000 μm (e.g., 20 μm, 50 μm, 100 μm, 150 μm, 200 μm, 250 μm, 300 μm, 350 μm, 400 μm, 450 μm, 500 μm, 550 μm, 600 μm, 650 μm, 700 μm, 750 μm, 800 μm, 850 μm, 900 μm, 950 μm, 1000 μm).

In some embodiments, the step of associating may include associating the outlined mechanoreceptors across the subset of images only if the outlined mechanoreceptors attributable to the same mechanoreceptor are present on at least a threshold number of consecutive images of the sequence of images. In some embodiments, the threshold number of the consecutive images is 2 to 20 (e.g., 2, 3, 4, 5, 6, 7, 8, 9, 10, 11, 12, 13, 14, 15, 16, 17, 18, 19, 20). In some embodiments, the threshold number of the consecutive images is 3.

In some embodiments, the step of associating comprises interpolating intermediate missing outlined mechanoreceptors in both size and location along an otherwise contiguous three-dimensional shape of the outlined mechanoreceptors. In some embodiments, the step of associating comprises interpolating intermediate missing boxes (which are false negatives in box detection) in both size and location along an otherwise contiguous MC (which consists of a group of coherent boxes across consecutive slices).

In another aspect, this disclosure also provides a system for determining one or more characteristics of mechanoreceptors in a region of interest on skin of a patient. In some embodiments, the system may include one or more processors configured to: (i) input image data comprising an image stack obtained from the region of interest on the skin of the patient; (ii) separate the stack image into a sequence of images; (iii) detect mechanoreceptors on each of the sequence of images and outline the detected mechanoreceptors (for example, with boxes) on each of the sequence of images using a trained annotator; (iv) associate each of the outlined mechanoreceptors (for example, associating boxes) on an image of the sequence of images with another outlined mechanoreceptor on a neighboring image of the sequence of images to reconstitute three-dimensional shapes of the outlined mechanoreceptors across a subset of the images; and (v) determine, based on the three-dimensional shapes of the outlined mechanoreceptors, one or more characteristics of the mechanoreceptors in the region of interest of the skin of the patient, wherein the one or more characteristics may include count, density, size, morphology, or a combination thereof.

In some embodiments, the system may include an imaging device for obtaining the image data. In some embodiments, the imaging device may include a confocal microscope. In some embodiments, the confocal microscope is an in vivo reflectance confocal microscope.

In some embodiments, the step of detecting mechanoreceptors is performed by a YOLOv4 object detection model. In some embodiments, the step of outlining may include placing bounding boxes around the outlined mechanoreceptors.

In some embodiments, the step of associating is performed based on intersection over union across the subset of the images.

In some embodiments, the step of associating may include associating the outlined mechanoreceptors across the subset of images only if the outlined mechanoreceptors attributable to the same mechanoreceptor are present on at least a threshold number of consecutive images of the sequence of images. In some embodiments, the threshold number of the consecutive images is 2 to 20. In some embodiments, the threshold number of the consecutive images is 3.

In some embodiments, the sequence of images may include 10 to 50 images (e.g., 35 images). In some embodiments, two neighboring images of the sequence of images are about 2 μm to 20 μm (e.g., 7 μm) apart.

In some embodiments, the mechanoreceptors may include Meissner's corpuscles. In some embodiments, the step of determining the one or more characteristics of the mechanoreceptors may include quantifying a density of Meissner's corpuscles. In some embodiments, the step of determining the one or more characteristics of the mechanoreceptors may include quantifying a size of Meissner's corpuscles.

In some embodiments, the one or more processors are further configured to determine the one or more characteristics of the mechanoreceptors over time to monitor a change in the one or more characteristics of the mechanoreceptors.

In some embodiments, the image data is obtained by confocal microscopy. In some embodiments, the confocal microscopy may include in vivo reflectance confocal microscopy (RCM).

### Methods for Characterization Mechanoreceptors for Monitoring a Condition in a Subject

In another aspect, this disclosure also provides a method of determining or monitoring a condition in the patient based on one or more characteristics of mechanoreceptors in a region of interest on skin of a patient. In some embodiments, the method may include determining one or more characteristics of mechanoreceptors in the region of interest on the skin of the patient according to the method described herein; and determining a condition in the patient based on the determined one or more characteristics of the mechanoreceptors.

In yet another aspect, this disclosure provides a system of determining or monitoring a condition in the patient based on one or more characteristics of mechanoreceptors in a region of interest on skin of a patient. In some embodiments, the system may include one or more processors configured to: determine one or more characteristics of mechanoreceptors in the region of interest on the skin of the patient according to the system described herein; and determine a condition in the patient based on the determined one or more characteristics of the mechanoreceptors.

In some embodiments, the condition may include a peripheral nervous system disorder. In some embodiments, the peripheral nervous system disorder may include sensory neuropathy. Sensory neuropathy can have many causes, including, but not limit to: immune-mediated (such as lupus, rheumatoid arthritis, Sjogren syndrome, celiac disease, and systemic lupus erythematosus; metabolic (such as diabetes and hyperlipidemia); nutritional deficiencies (such as vitamin B12, copper, vitamin E, and folic acid); toxic (such as chemotherapy, drug-induced, and alcohol); hereditary (such as hemochromatosis, Fabry disease, Ehlers-Danlos syndrome, and Friedreich ataxia); infectious (such as syphilis, leprosy, HIV, hepatitis C, and cryoglobulinemia); and other (such as fibromyalgia and vasculitis). In some embodiments, the sensory neuropathy is caused by diabetes. In some embodiments, the sensory neuropathy is caused by HIV.

In some embodiments, the mechanoreceptors may include MCs.

In some embodiments, one or more features may include location within tips of dermal papillae of glabrous skin, absence of similar profiles in hairy skin, dimensions in a range of reference dimensions of MCs in skin biopsy specimens, orientation with a long axis perpendicular to a dermal-epidermal junction, presence of one to two MCs per-dermal papilla, presence of an encapsulated structure with an internal lobulated axonal architecture, or a combination thereof.

As used herein, the term “monitoring” refers to monitoring or assessing recovery of sensory innervation (nerve supply) to the skin following peripheral nerve surgery or repair or nonsurgical treatments. For example, if a patient has a nerve traumatized in a limb and a surgeon repaired it, or did a nerve graft, one could monitor recovery by monitoring the improvement in density (or lack thereof) of MCs in the relevant region of skin on the hand or foot. Another very common instance may be that of carpal tunnel syndrome (compressed median nerve at the wrist). MCs potentially could be imaged and counted to assess the severity of carpal tunnel syndrome, and also to assess the degree of recovery following carpal tunnel release surgery. Neuropathies, such as the median neuropathy at the wrist that produces carpal tunnel syndrome or other traumatic neuropathies, are focal neuropathies or mononeuropathies, as distinct from the more generalized peripheral neuropathies associated with, for example, diabetes. These instances and uses (e.g., for assessing recovery) are covered by the term “monitoring.” The term “monitoring” also includes looking for progressive decline, indicating disease progression. Also, the term “peripheral nervous system disorders” should encompass both generalized peripheral neuropathies and neuropathies affecting a single nerve. In contrast, assessing whether a Meissner corpuscle density is normal or markedly reduced is very helpful in making this differentiation.

In some embodiments, the step of determining one or more characteristics of the mechanoreceptors may include quantifying the density of the MCs. In some embodiments, the step of determining one or more characteristics of the mechanoreceptors may include quantifying the size of the MCs. In some embodiments, the step of identifying the mechanoreceptors and the step of determining the one or more characteristics of the mechanoreceptors are performed over time to monitor a change in the one or more characteristics of the mechanoreceptors.

In some embodiments, the image data is obtained by confocal microscopy. In some embodiments, the confocal microscopy may include in vivo reflectance confocal microscopy (RCM).

In some embodiments, the system may include an imaging device for obtaining the image data. In some embodiments, the imaging device may include a confocal microscope. In some embodiments, the confocal microscope is an in vivo reflectance confocal microscope.

In some embodiments, the disclosed methods may be used for prognosis or predicting responsiveness to a therapy.

The terms “determining responsiveness,” “predicting responsiveness,” and “assessing a likelihood of a therapeutic response” may be used interchangeably herein.

The term “prognosis,” as used herein, refers to anticipation of progression of a disease (e.g., a peripheral nervous system disorder) or condition and prospect (e.g., the probability, duration, and/or extent) of recovery. A good prognosis of the diseases or conditions may generally encompass anticipation of a satisfactory partial or complete recovery from the diseases or conditions, such as within an acceptable time period. A good prognosis of such may more commonly encompass anticipation of not further worsening or aggravating within a given time period. A poor prognosis of the diseases or conditions as taught herein may generally encompass anticipation of a substandard recovery and/or unsatisfactorily slow recovery, or to substantially no recovery or even further worsening of such.

In some embodiments, a therapeutic response may include a response when referring to a patient treated with a therapy for a peripheral nervous system disorder. For example, a response may include at least one positive therapeutic effect, such as a reduced symptom of a peripheral nervous system disorder.

The terms “predicting,” “prediction,” or “predictive,” as used herein, refer to an advance declaration, indication, or foretelling of a response or reaction to a therapy in a subject not (yet) having been treated with the therapy. For example, a prediction of responsiveness (or sensitivity or susceptibility) to a therapy in a subject may indicate that the subject will respond or react to the therapy, for example, within a certain time period, e.g., so that the subject will have a clinical benefit from the therapy. A prediction of unresponsiveness (or insensitivity or insusceptibility) to a therapy in a subject may indicate that the subject will minimally or not respond or react to the therapy, for example, within a certain time period, e.g., so that the subject will have no clinical benefit from the therapy.

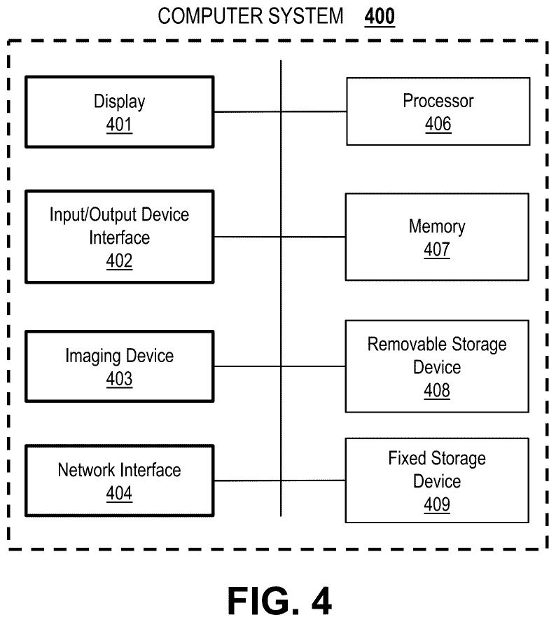

FIG. 4 is a functional diagram illustrating a programmed computer system in accordance with some embodiments. As will be apparent, other computer system architectures and configurations can be used to perform the described methods. Computer system 400, which includes various subsystems as described below, includes at least one microprocessor subsystem (also referred to as a processor or a central processing unit (CPU) 406). For example, processor 406 can be implemented by a single-chip processor or by multiple processors. In some embodiments, processor 406 is a general-purpose digital processor that controls the operation of the computer system 400. In some embodiments, processor 406 also includes one or more coprocessors or special purpose processors (e.g., a graphics processor, a network processor, etc.). Using instructions retrieved from memory 407, processor 406 controls the reception and manipulation of input data received on an input device (e.g., image processing device 403, I/O device interface 402), and the output and display of data on output devices (e.g., display 401).

Processor 406 is coupled bi-directionally with memory 407, which can include, for example, one or more random access memories (RAM) and/or one or more read-only memories (ROM). As is well known in the art, memory 407 can be used as a general storage area, a temporary (e.g., scratchpad) memory, and/or a cache memory. Memory 407 can also be used to store input data and processed data, as well as to store programming instructions and data, in the form of data objects and text objects, in addition to other data and instructions for processes operating on processor 406. Also, as is well known in the art, memory 407 typically includes basic operating instructions, program code, data, and objects used by the processor 406 to perform its functions (e.g., programmed instructions). For example, memory 407 can include any suitable computer-readable storage media described below, depending on whether, for example, data access needs to be bi-directional or uni-directional. For example, processor 406 can also directly and very rapidly retrieve and store frequently needed data in a cache memory included in memory 407.

A removable mass storage device 408 provides additional data storage capacity for the computer system 400 and is optionally coupled either bi-directionally (read/write) or uni-directionally (read-only) to processor 406. A fixed mass storage 409 can also, for example, provide additional data storage capacity. For example, storage devices 408 and/or 409 can include computer-readable media such as magnetic tape, flash memory, PC-CARDS, portable mass storage devices such as hard drives (e.g., magnetic, optical, or solid-state drives), holographic storage devices, and other storage devices. Mass storages 408 and/or 409 generally store additional programming instructions, data, and the like that typically are not in active use by the processor 406. It will be appreciated that the information retained within mass storages 408 and 409 can be incorporated, if needed, in a standard fashion as part of memory 407 (e.g., RAM) as virtual memory.

In addition to providing processor 406 access to storage subsystems, bus 410 can be used to provide access to other subsystems and devices as well. As shown, these can include a display 401, a network interface 404, an input/output (I/O) device interface 402, an image processing device 403, as well as other subsystems and devices. For example, image processing device 403 can include a camera, a scanner, etc.; I/O device interface 402 can include a device interface for interacting with a touchscreen (e.g., a capacitive touch sensitive screen that supports gesture interpretation), a microphone, a sound card, a speaker, a keyboard, a pointing device (e.g., a mouse, a stylus, a human finger), a global positioning system (GPS) receiver, a differential global positioning system (DGPS) receiver, an accelerometer, and/or any other appropriate device interface for interacting with system 400. Multiple I/O device interfaces can be used in conjunction with computer system 400. The I/O device interface can include general and customized interfaces that allow the processor 406 to send and, more typically, receive data from other devices such as keyboards, pointing devices, microphones, touchscreens, transducer card readers, tape readers, voice or handwriting recognizers, biometrics readers, cameras, portable mass storage devices, and other computers.

The network interface 404 allows processor 406 to be coupled to another computer, computer network, or telecommunications network using a network connection as shown. For example, through the network interface 404, the processor 406 can receive information (e.g., data objects or program instructions) from another network, or output information to another network in the course of performing method/process steps. Information, often represented as a sequence of instructions to be executed on a processor, can be received from and outputted to another network. An interface card or similar device and appropriate software implemented by (e.g., executed/performed on) processor 406 can be used to connect the computer system 400 to an external network and transfer data according to standard protocols. For example, various process embodiments disclosed herein can be executed on processor 406 or can be performed across a network such as the Internet, intranet networks, or local area networks, in conjunction with a remote processor that shares a portion of the processing. Additional mass storage devices (not shown) can also be connected to processor 406 through network interface 404.

In addition, various embodiments disclosed herein further relate to computer storage products with a computer-readable medium that includes program code for performing various computer-implemented operations. The computer-readable medium includes any data storage device that can store data that can thereafter be read by a computer system. Examples of computer-readable media include, but are not limited to: magnetic media such as disks and magnetic tape; optical media such as CD-ROM disks; magneto-optical media such as optical disks; and specially configured hardware devices such as application-specific integrated circuits (ASICs), programmable logic devices (PLDs), and ROM and RAM devices. Examples of program code include both machine code as produced, for example, by a compiler, or files containing higher level code (e.g., script) that can be executed using an interpreter.

The computer system as shown in FIG. 4 is an example of a computer system suitable for use with the various embodiments disclosed herein. Other computer systems suitable for such use can include additional or fewer subsystems. In some computer systems, subsystems can share components (e.g., for touchscreen-based devices such as smartphones, tablets, etc., I/O device interface 402 and display 401 share the touch-sensitive screen component, which both detects user inputs and displays outputs to the user). In addition, bus 410 is illustrative of any interconnection scheme serving to link the subsystems. Other computer architectures having different configurations of subsystems can also be utilized.

### Additional Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

Aspects of the present disclosure are described herein with reference to flowchart illustrations and/or block diagrams of methods, apparatus (systems), and computer program products according to embodiments of the invention. In some embodiments, the flowchart and block diagrams in the Figures illustrate the architecture, functionality, and operation of possible implementations of systems, methods, and computer program products according to various embodiments of the present invention. In this regard, each block in the flowchart or block diagrams may represent a module, a segment, or a portion of instructions, which may include one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

These computer readable program instructions may be provided to a processor of a general-purpose computer, a special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein may include an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

It will be understood that, although the terms “first,” “second,” etc., may be used herein to describe various elements, components, regions, layers and/or sections. These elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of example embodiments.

Unless specifically stated otherwise, as apparent from the above discussion, it is appreciated that throughout the description, discussions utilizing terms such as “processing,” “performing,” “receiving,” “computing,” “calculating,” “determining,” “identifying,” “displaying,” “providing,” “merging,” “combining,” “running,” “transmitting,” or the like, refer to the action and processes of a computer system, or similar electronic computing device, that manipulates and transforms data represented as physical (or electronic) quantities within the computer system memories or registers or other such information storage, transmission or display devices.

As used herein, the term “if may be construed to mean “when” or “upon” or “in response to determining,” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

The terms or acronyms like “convolutional neural network,” “CNN,” “neural network,” “NN,” “deep neural network,” “DNN,” “recurrent neural network,” “RNN,” and/or the like may be interchangeably referenced throughout this document.

An “electronic device” or a “computing device” refers to a device that includes a processor and memory. Each device may have its own processor and/or memory, or the processor and/or memory may be shared with other devices as in a virtual machine or container arrangement. The memory will contain or receive programming instructions that, when executed by the processor, cause the electronic device to perform one or more operations according to the programming instructions.

The terms “memory,” “memory device,” “computer-readable medium,” “data store,” “data storage facility” and the like each refer to a non-transitory device on which computer-readable data, programming instructions or both are stored. Except where specifically stated otherwise, the terms “memory,” “memory device,” “computer-readable medium,” “data store,” “data storage facility” and the like are intended to include single device embodiments, embodiments in which multiple memory devices together or collectively store a set of data or instructions, as well as individual sectors within such devices.

The terms “processor” and “processing device” refer to a hardware component of an electronic device that is configured to execute programming instructions, such as a microprocessor or other logical circuit. A processor and memory may be elements of a microcontroller, custom configurable integrated circuit, programmable system-on-a-chip, or other electronic device that can be programmed to perform various functions. Except where specifically stated otherwise, the singular term “processor” or “processing device” is intended to include both single-processing device embodiments and embodiments in which multiple processing devices together or collectively perform a process.

In this document, the terms “communication link” and “communication path” mean a wired or wireless path via which a first device sends communication signals to and/or receives communication signals from one or more other devices. Devices are “communicatively connected” if the devices are able to send and/or receive data via a communication link. “Electronic communication” refers to the transmission of data via one or more signals between two or more electronic devices, whether through a wired or wireless network, and whether directly or indirectly via one or more intermediary devices.

It is noted here that, as used in this specification and the appended claims, the singular forms “a,” “an,” and “the” include plural reference unless the context clearly dictates otherwise.

As used herein, “plurality” means two or more. As used herein, a “set” of items may include one or more of such items.

As used herein, “including,” “comprising,” “containing,” or “having” and variations thereof are meant to encompass the items listed thereafter and equivalents thereof as well as additional subject matter unless otherwise noted.

As used herein, the phrases “in one embodiment,” “in various embodiments,” “in some embodiments,” and the like do not necessarily refer to the same embodiment, but may unless the context dictates otherwise.

As used herein, the terms “and/or” or “/” means any one of the items, any combination of the items, or all of the items with which this term is associated.

As used herein, the term “substantially” does not exclude “completely,” e.g., a composition which is “substantially free” from Y may be completely free from Y. Where necessary, the word “substantially” may be omitted from the definition of the present disclosure.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As used herein, the term “each,” when used in reference to a collection of items, is intended to identify an individual item in the collection but does not necessarily refer to every item in the collection. Exceptions can occur if explicit disclosure or context clearly dictates otherwise.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the present disclosure. The upper and lower limits of these smaller ranges may independently be included or excluded in the range, and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

The use of any and all examples, or exemplary language (e.g., “such as”) provided herein, is intended merely to better illuminate the invention and does not pose a limitation on the scope of the present disclosure unless otherwise claimed. No language in the specification should be construed as indicating any non-claimed element as essential to the practice of the present disclosure.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise. In cases in which a method may include a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present disclosure. Nothing herein is to be construed as an admission that the present disclosure is not entitled to antedate such publication by virtue of prior disclosure. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1

In vivo Confocal Microscopy Procedure.

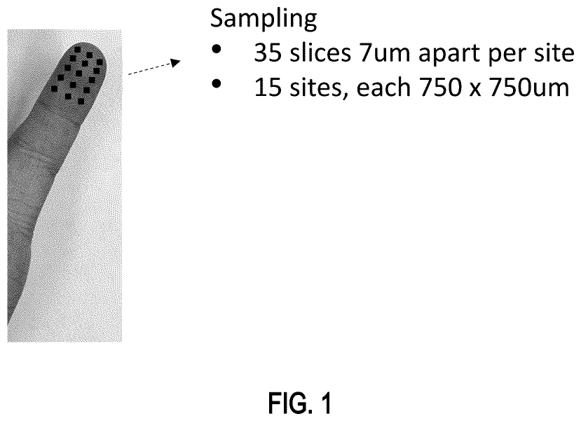

A trained technician performed in vivo RCM within a standardized 3×3 mm skin area over the mid-point of the volar aspect of the distal phalanx of digit V and over the midpoint of the thenar eminence of the non-dominant hand in each subject. An in vivo RCM, (VivaScope 3000, Inc, Rochester, NY) was used for all imaging. In order to determine whether MCs could be visualized with in vivo RCM, the technician performed the imaging through a microscope to skin contact device. A drop of Crodamol STS (Croda, USA), an emollient ester, is placed on the skin. Using a 30× immersible objective lens and ultrasound gel, matrices of 0.75 mm×0.75 mm images were acquired in progressively deeper horizontal planes through the dermis at each skin site. The most superficial matrix of images was at a plane through the top of the dermal papillae, where the most superficial MCs were visualized. Up to 36 additional matrices of images were acquired, each in a plane deeper than the prior, to extend through the entire depth of the dermal layer. The matrices consisted of between 15 to 30 evenly spaced 0.75 mm×0.75 mm images. Imaging site specific templates were adhered to subjects using predefined anatomical landmarks in order to standardize imaging locations (see, e.g., FIG. 1).

### Data Annotation and Model Training

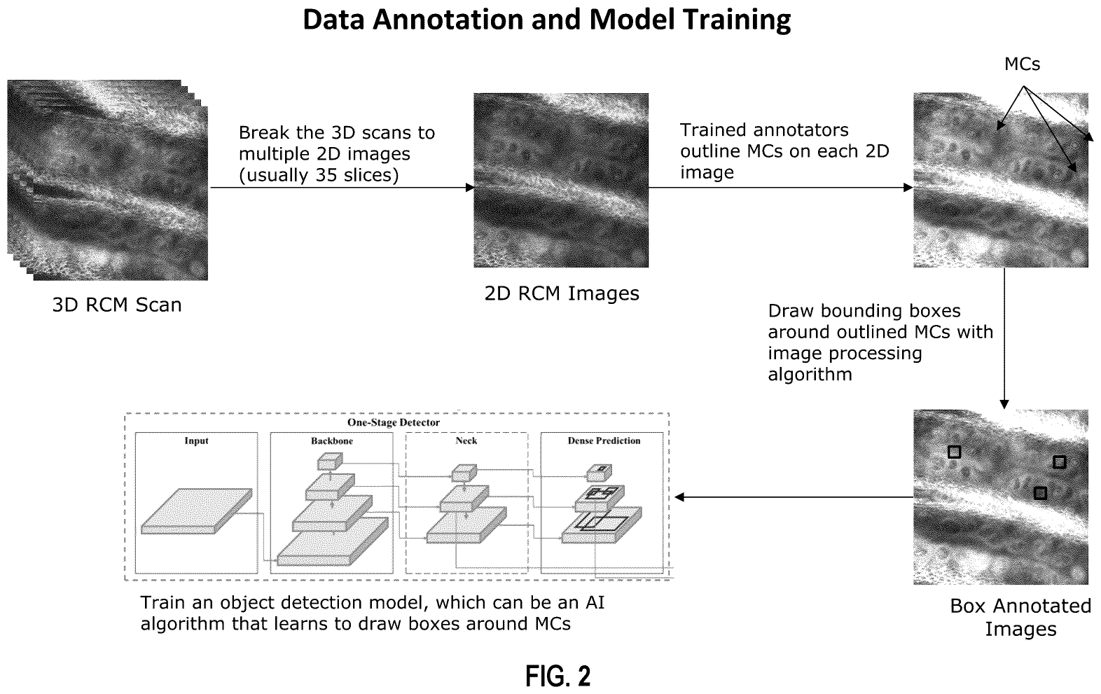

As shown in FIG. 2, a 3D RCM scan was broken into multiple 2D RCM images (usually 35 slices). Next, trained human annotators were used to outline MCs on every 2D image. Bounding boxes were then drawn around outlined MCs with a straightforward image processing algorithm. An object detection model based on YOLOv4 (M. Ning, et al., 2021 IEEE 45th Annual Computers, Software, and Applications Conference (COMPSAC), Madrid, Spain, 2021, pp. 31-36) was subsequently trained with the box-annotated images to automatically draw boxes around MCs in 2D RCM images in an unseen 3D RCM scan volume.

### Prediction and Postprocessing

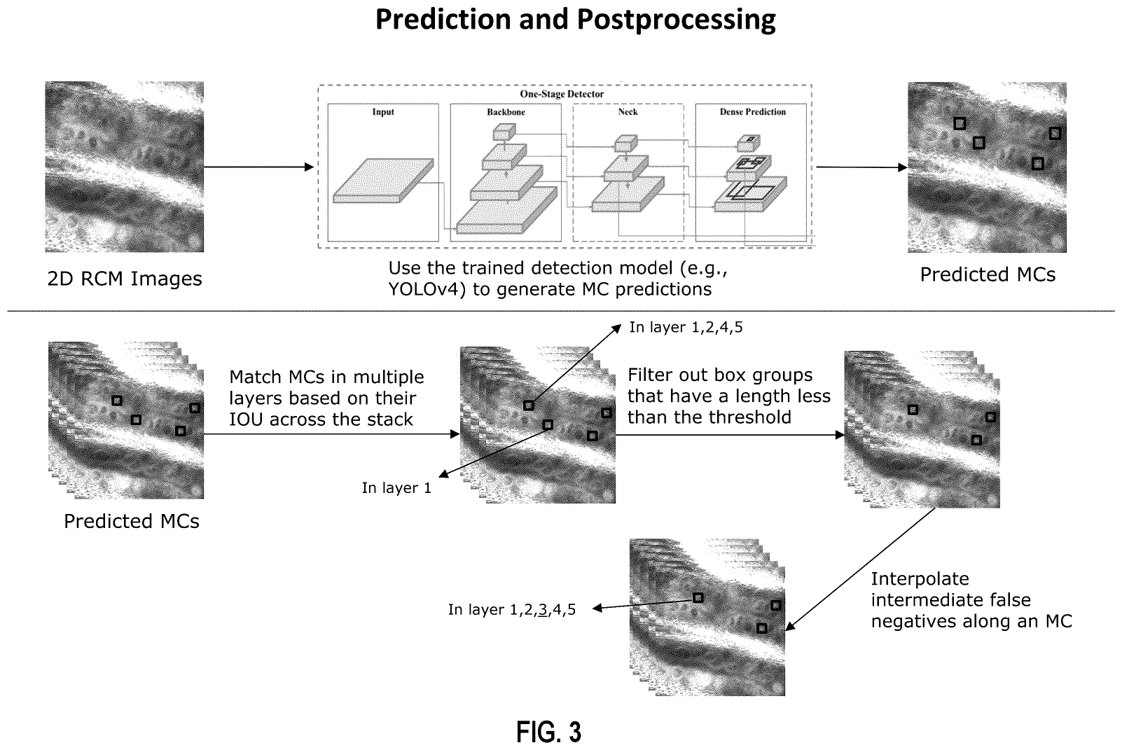

As shown in FIG. 3, the 3D RCM images were sliced into 2D RCM images, and the trained YOLOv4 detection model was used to generate MC box predictions. A box was retained if the detection confidence given by the YOLOv4 model was over 0.4. The MC boxes in multiple layers were matched based on their IOU between neighboring slices across the stack. After matching, the box groups that have a length less than a pre-defined threshold were filtered out. Finally, the intermediate missing boxes (which are false negatives in box detection) were interpolate in both size and location along an otherwise contiguous MC (which consists of a group of coherent boxes across consecutive slices).

### Example 2

### Automated MC Quantification Using AI

### Step 1: Object Detection Model Identifies MCs on 2D Images

detections count=501, unique truth count=87
classid=0, name=Meissner_corpuscle, ap=78.86% (TP=69, FP=25)
for confthresh=0.25, precision=0.73, recall=0.79, Fl-score=0.76
for confthresh=0.25, TP=69, FP=25, FN=18, average IoU=55.44%
IoU threshold=50%, used Area-Under-Curve for each unique Recall
mean average precision (mAP@0.50)=0.788626, or 78.86%
Total Detection Time: 10 Seconds

### Step 2: Post-Processing Algorithm Uses 3D Information to Count MCs Per Stack.

TABLE 1
MC identification and prediction

| Stack | 2D Unique Ground | 2D Unique | Ground Truth | Prediction |
|---|---|---|---|---|
| ID | Truth | Predictions | MCs | MCs |
| 1 | 12 | 14 | 1 | 1 |
| 2 | 19 | 26 | 3 | 3 |
| 3 | 16 | 15 | 2 | 2 |
| 4 | 40 | 44 | 5 | 6 |

Table 2 summarizes the results of the two phases of training (all with a 0.4 confidence threshold).

TABLE 2
Results of two phases training

| Phase | Train | Val | Test |
|---|---|---|---|
| 1 | 88.07 | 84.35 | 84.35 |
| 2-AII | 90.26 | 87.11 | 83.69 |
| 2-FP | 90.45 | 88.49 | 82.08 |
| 2-FN | 90.62 | 83.64 | 83.21 |

The present disclosure is not to be limited in scope by the specific embodiments described herein. Indeed, various modifications of the invention, in addition to those described herein, will become apparent to those skilled in the art from the foregoing description and the accompanying figures. Such modifications are intended to fall within the scope of the appended claims.

What is claimed is:

1. A method for determining one or more characteristics of mechanoreceptors in a region of interest on skin of a patient, comprising:
inputting image data comprising an image stack obtained from the region of interest on the skin of the patient;
separating the stack image into a sequence of images;
detecting mechanoreceptors on each of the sequence of images and outlining the detected mechanoreceptors on each of the sequence of images using a trained annotator;
associating each of the outlined mechanoreceptors on an image of the sequence of images with another outlined mechanoreceptor on a neighboring image of the sequence of images to reconstitute three-dimensional shapes of the outlined mechanoreceptors across a subset of the images; and
determining, based on the three-dimensional shapes of the outlined mechanoreceptors, one or more characteristics of the mechanoreceptors in the region of interest of the skin of the patient, wherein the one or more characteristics comprise count, density, size, morphology, or a combination thereof.

2. The method of claim 1, wherein the step of detecting mechanoreceptors is performed by an object detection model.

3. The method of claim 1, wherein the step of outlining comprises placing bounding boxes around the outlined mechanoreceptors.

4. The method of claim 1, wherein the step of associating is performed based on intersection over union across the subset of the images.

5. The method of claim 1, wherein the step of associating comprises:
(i) filtering out the associated outlined mechanoreceptors that are shorter than a threshold length;
(ii) associating the outlined mechanoreceptors across the subset of images only if the outlined mechanoreceptors attributable to the same mechanoreceptor are present on at least a threshold number of consecutive images of the sequence of images; or
(iii) interpolating intermediate missing outlined mechanoreceptors in both size and location along an otherwise contiguous three-dimensional shape of the outlined mechanoreceptors.

6. The method of claim 5, wherein the threshold number of the consecutive images is 2 to 20.

7. The method of claim 1, wherein the sequence of images comprise 10 to 50 images.

8. The method of claim 1, wherein the mechanoreceptors comprise Meissner's corpuscles.

9. The method of claim 8, wherein the step of determining the one or more characteristics of the mechanoreceptors comprises quantifying a density or a size of the Meissner's corpuscles.

10. The method of claim 1, wherein the image data is obtained by confocal microscopy.

11. A method of determining or monitoring a condition in the patient based on one or more characteristics of mechanoreceptors in a region of interest on skin of a patient, comprising:
determining one or more characteristics of mechanoreceptors in the region of interest on the skin of the patient according to the method of any one of the preceding claims; and
determining a condition in the patient based on the determined one or more characteristics of the mechanoreceptors.

12. A system for determining one or more characteristics of mechanoreceptors in a region of interest on skin of a patient, comprising one or more processors configured to:
input image data comprising an image stack obtained from the region of interest on the skin of the patient;
separate the stack image into a sequence of images;
detect mechanoreceptors on each of the sequence of images and outline the detected mechanoreceptors on each of the sequence of images using a trained annotator;
associate each of the outlined mechanoreceptors on an image of the sequence of images with another outlined mechanoreceptor on a neighboring image of the sequence of images to reconstitute three-dimensional shapes of the outlined mechanoreceptors across a subset of the images; and
determine, based on the three-dimensional shapes of the outlined mechanoreceptors, one or more characteristics of the mechanoreceptors in the region of interest of the skin of the patient, wherein the one or more characteristics comprise count, density, size, morphology, or a combination thereof.

13. The system of claim 12, wherein the step of outlining comprises placing bounding boxes around the outlined mechanoreceptors.

14. The system of claim 12, wherein the step of associating is performed based on intersection over union across the subset of the images.

15. The method of claim 12, wherein the step of associating comprises:
(i) filtering out the associated outlined mechanoreceptors that are shorter than a threshold length;
(ii) associating the outlined mechanoreceptors across the subset of images only if the outlined mechanoreceptors attributable to the same mechanoreceptor are present on at least a threshold number of consecutive images of the sequence of images; or
(iii) interpolating intermediate missing outlined mechanoreceptors in both size and location along an otherwise contiguous three-dimensional shape of the outlined mechanoreceptors.

16. The system of claim 15, wherein the threshold number of the consecutive images is 2 to 20.

17. The system of claim 12, wherein the sequence of images comprise 10 to 50 images.

18. The system of claim 12, wherein the mechanoreceptors comprise Meissner's corpuscles.

19. The system of claim 18, wherein the step of determining the one or more characteristics of the mechanoreceptors comprises quantifying a density or a size of the Meissner's corpuscles.

20. A system of determining or monitoring a condition in the patient based on one or more characteristics of mechanoreceptors in a region of interest on skin of a patient, comprising one or more processors configured to:
determine one or more characteristics of mechanoreceptors in the region of interest on the skin of the patient according to the system of claim 12; and
determine a condition in the patient based on the determined one or more characteristics of the mechanoreceptors.
